(19) United States
(12) Patent Application Publication
(10) Pub. No.: US 2007/0179513 A1
(43) Pub. Date: Aug. 2, 2007

(54) Method and device for removing an occlusion

(76) Inventor: Harvey L. Deutsch, Los Angeles, CA (US)

Correspondence Address: SHELDON MAK ROSE & ANDERSON PC, 100 East Corson Street, Third Floor, PASADENA, CA, 91103-3842, US

(21) Appl. No.: 10/566,853

(22) PCT Filed: Dec. 23, 2005
(86) PCT No.: PCT/US05/47039

§ 371 Date: Jan. 31, 2006

Related U.S. Application Data
(63) Continuation-in-part of application No. PCT/US05/00508, filed on Jan. 7, 2005.
(60) Provisional application No. 60/638,597, filed on Dec. 23, 2004.
(60) Provisional application No. 60/535,224, filed on Jan. 9, 2004.
(60) Provisional application No. 60/563,597, filed on Apr. 19, 2004.

(51) Int. Cl. A61B 17/22

(52) U.S. Cl. 606/159

(57) ABSTRACT

A device for removing an occlusion from within the central lumen of a tubular structure, where the device comprises an occlusion removing structure having a low profile configuration and having a high profile configuration. A method for removing an occlusion from within the central lumen of a tubular structure, where the method comprises providing a device according to the present invention.

### CROSS-REFERENCE TO RELATED APPLICATION

The present Application claims the benefit of U.S. Provisional Patent Application 60/638,597 titled “Cleaner for Tubular Structure,” filed Dec. 23, 2004, and claims priority from International Patent Application No. PCT/US05/00508, filed Jan. 7, 2005, the contents of which are incorporated in this disclosure by reference in their entirety.

### BACKGROUND

There are a large variety of tubular structures having a central axial lumen used in medical applications, and in non-medical commercial and industrial applications. Examples of tubular structures used in medical applications include catheters, drains and tubes. In many such applications, the tubular structure is prone to collecting debris within the central lumen of the tubular structure that occludes the tubular structure, causing a decrease or cessation of function of the tubular structure, and necessitating that the occlusion be removed or that the tubular structure be replaced.

In medical applications in particular, replacing a tubular structure can be very costly, and can put a patient at increased risk of infection. Therefore, it is often preferable to remove the occlusion from within the central lumen of the tubular structure. In some applications, the occlusion can be removed by flushing the central lumen of the tubular structure with a liquid, thereby moving the occlusion distally. In many applications, however, flushing the occlusion distally is not acceptable, such as, for example, when using a tubular structure to drain a cavity or space within the human body.

Therefore, there is a need for a method of removing an occlusion from within the central lumen of a tubular structure, such as a tubular structure used in a medical application, that does not comprise flushing the occlusion distally.

### SUMMARY

According to one embodiment of the present invention, there is provided a device for removing an occlusion from within the central lumen of a tubular structure. In one embodiment the device comprises a proximal end, a distal end and an intermediate segment between the proximal end and the distal end; and comprises an occlusion removing structure having a low profile configuration and having a high profile configuration, the occlusion removing structure comprising a proximal end, a distal end and an intermediate segment between the proximal end and the distal end, and comprising an outer diameter.

In a preferred embodiment, the occlusion removing structure comprises an extension of the intermediate segment; and the occlusion removing structure further comprises one or more than one foldable disc mounted transversely on the extension, and converting the device from the low profile configuration to the high profile configuration comprises unfolding the one or more than one disc; where the device further comprises a sheath comprising a proximal end, a distal end, and a central lumen; where the sheath surrounds the intermediate segment of the occlusion removing structure, and where the sheath is axially movable with respect to the occlusion removing structure. In one embodiment, the one or more than one foldable disc is a plurality of discs. In another embodiment, at least one of the plurality of discs has a diameter different from another of the plurality of discs. In one embodiment, the one or more than one foldable disc is at least three discs, and the distance between two adjacent discs is different from the distance between two different adjacent discs. In one embodiment, each disc comprises silicone.

In a preferred embodiment, the device further comprises a sheath comprising a proximal end, a distal end, and a central lumen; where the sheath is axially movable with respect to the occlusion removing structure; where the distal end of the occlusion removing structure comprises a self-expanding portion; where the sheath functions to compress the self expanding portion within the central lumen of the sheath; and where converting the device from the low profile configuration to the high profile configuration comprises relieving the ,compression on the self-expanding portion. In one embodiment, the distal end of the sheath further comprises a flaring distal collar configured to permit the self-expanding portion of the distal end of the device to be retracted into the central lumen of the sheath.

In one embodiment, the device, further comprises a removable tab surrounding the intermediate segment of the occlusion removing structure, immediately distal to the proximal end of the device, where the tab. prevents extrusion of the distal end of the occlusion removing structure from the distal end of the sheath. In another embodiment, the sheath comprises a proximal collar configured to prevent the proximal end of the occlusion removing structure from entering the central lumen of the sheath. In one embodiment, the occlusion removing structure further comprises a mechanism for applying axial force to the distal end of the occlusion removing structure. In another embodiment, the device further comprises an enclosure bag comprising a proximal end, a distal end and an intermediate segment, where the intermediate segment of the enclosure bag comprises a tube of material capable of being reversibly compressed and extended axially.

In another preferred embodiment, the distal end of the occlusion removing structure comprises an inflatable balloon, and converting the device from the low profile configuration to the high profile configuration comprises inflating the balloon; and the device further comprises an enclosure bag comprising a proximal end, a distal end and an intermediate segment, where the intermediate segment of the enclosure bag comprises a tube of material capable of being reversibly compressed and extended axially. In another preferred embodiment, the device has a long axis, the distal end of the occlusion removing structure comprises an expandable wire basket, and converting the device from the low profile configuration to the high profile configuration comprises activating a mechanism, thereby transmitting axial force to contract the wire basket in a distal to proximal direction, and thereby expanding the wire basket in a direction perpendicular to the axis of the device; and where the device further comprises an enclosure bag comprising a proximal end, a distal end and an intermediate segment, where the intermediate segment of the enclosure bag comprises a tube of material capable of being reversibly compressed and extended axially.

In one embodiment, the intermediate segment of the enclosure bag comprises a substantially transparent material. In another embodiment, the proximal end of the enclosure bag comprises a proximal end piece comprising an axial lumen having a diameter greater than the outer diameter of the occlusion removing structure. In another embodiment, the distal end of the enclosure bag comprises a distal end piece comprising an axial lumen having a diameter greater than the outer diameter of the occlusion removing structure. In another embodiment, the distal end of the enclosure bag comprises a connector for mating with the proximal end of the tubular structure with an occlusion. In a preferred embodiment, the connector is a “Christmas Tree” type connector or is a Luer-lock type connector. In one embodiment, the enclosure bag further comprises a removable cover.

In one embodiment, the proximal end of the device further comprises a connector. In another embodiment, the occlusion removing structure comprises an extension of the intermediate segment, and the extension comprises a blunted tip at the distal end of the occlusion removing structure.

According to one embodiment of the present invention, there is provided a method for removing an occlusion from a tubular structure, where the tubular structure comprises a proximal end, a distal end, an intermediate segment and a central lumen within the intermediate segment, and where the occlusion is within the central lumen. The method comprises a) providing means for removing an occlusion from within the central lumen of a tubular structure, where the means for removing an occlusion comprises a proximal end and a distal end, and comprises an occlusion removing structure having a low profile configuration and having a high profile configuration; b) inserting the distal end of the means for removing an occlusion into the proximal end of the tubular structure in the low profile configuration; c) advancing the means for removing an occlusion through the central lumen of the tubular structure until the distal end of the means for removing an occlusion is at least partly distal to the occluding material; d) converting the occlusion removing structure into the high profile configuration; and e) withdrawing the means for removing an occlusion. from the tubular structure while the occlusion removing structure is in the high profile configuration, thereby removing some or all of the occluding material from the tubular structure. In one embodiment, the means for removing an occlusion comprises one or more than one foldable disc, and converting the occlusion removing structure into the high profile configuration comprises unfolding the one or more than one foldable disc. In another embodiment, the means for removing an occlusion comprises a self-expanding portion, and where converting the occlusion removing structure into the high profile configuration comprises relieving compressive force on the self-expanding portion. In another embodiment, the means for removing an occlusion comprises an enclosure bag; and withdrawing the means for removing an occlusion causes the occluding material to deposit within the enclosure bag. In one embodiment, the means for removing an occlusion comprises an inflatable balloon; converting the occlusion removing structure into the high profile configuration comprises inflating the inflatable balloon; the means for removing an occlusion comprises an enclosure bag; and withdrawing the means for removing an occlusion causes the occluding material to deposit within the enclosure bag. In one embodiment, the means for removing an occlusion comprises a wire basket; converting the occlusion removing structure into the high profile configuration comprises activating a mechanism to transmit axial force to contract a wire basket in a distal to proximal direction thereby expanding the wire basket; the means for removing an occlusion comprises an enclosure bag; and withdrawing the means for removing an occlusion causes the occluding material to deposit within the enclosure bag. In a preferred embodiment, the method comprises repeating steps b) through e). In another embodiment, the means for removing an occlusion is a first means for removing an occlusion, and the method further comprises performing the steps b) through e) with a second means for removing an occlusion, where the first means for removing an occlusion and the second means for removing an occlusion comprise different mechanisms for converting the occlusion removing structure from the low profile configuration to the high profile configuration. In another embodiment, the tubular structure comprises a connector on the proximal end, and the method further comprises connecting the means for removing an occlusion to the connector.

According to one embodiment of the present invention, there is provided a method for removing an occlusion from a tubular structure, where the tubular structure comprises a proximal end, a distal end, an intermediate segment and a central lumen within the intermediate segment, and where the occlusion is within the central lumen. The method comprises a) providing a device according to the present invention; b) inserting the distal end of the device into the proximal end of the tubular structure in the low profile configuration; c) advancing the device through the central lumen of the tubular structure until the distal end of the device is at least partly distal to the occluding material; d) converting the occlusion removing structure into the high profile configuration; and e) withdrawing the device from the tubular structure while the occlusion removing structure is in the high profile configuration, thereby removing the occluding material from the tubular structure. In one embodiment, the device comprises an enclosure bag, and withdrawing the device causes the occluding material to deposit within the enclosure bag. In another embodiment, the device further comprises a removable tab; and the method further comprises removing the tab from the intermediate segment, thereby permitting the distal end of the occlusion removing structure to extrude through the distal end of the sheath. In one embodiment, withdrawing the device causes the occluding material to deposit within the enclosure bag. In another embodiment, the method comprises repeating steps b) through e). In one embodiment, the device is a first device, and the method further comprises performing the steps b) through e) with a second device, where the first device and the second device comprise different mechanisms for converting the occlusion removing structure from the low profile configuration to the high profile configuration. In another embodiment, the tubular structure comprises a connector on the proximal end, and the method further comprises connecting the device to the connector. In another embodiment, the method further comprises unconnecting the connector from the device after removing the occluding material.

### FIGURES

These and other features, aspects and advantages of the present invention will become better understood with regard to the following description, appended claims, and accompanying figures where:

FIG. 1 is a lateral perspective view of one embodiment of a device for removing an occlusion from within the central lumen of a tubular structure according to the present invention including the optional enclosure bag;

FIG. 2 is a lateral perspective view of the occlusion removing structure of the device shown in FIG. 1;

FIG. 3 is an enlarged cutaway lateral perspective view of the distal end of the device shown in FIG. 1;

FIG. 4 is an enlarged lateral perspective view of the distal end of the occlusion removing structure of the device shown in FIG. 1, FIG. 2 and FIG. 3;

FIG. 5 is a lateral perspective view of an enclosure bag of the device according to the present invention;

FIG. 6 is a lateral perspective view of another embodiment of a device for removing an occlusion from within the central lumen of a tubular structure according to the present invention;

FIG. 7 is a partial lateral perspective view of the device shown in FIG. 6 with the distal end of the occlusion removing structure extruded from the distal end of the sheath;

FIG. 8. is an enlarged cutaway partial lateral perspective. view of the device shown in FIG. 6 including the optional enclosure bag;

FIG. 9 is a lateral perspective view of another embodiment of a device for removing an occlusion from within the central lumen of a tubular structure according to the present invention in the low profile configuration;

FIG. 10 is a close-up lateral perspective view of the distal end of the device shown in FIG. 9 in the high profile configuration;

FIG. 11 is a lateral perspective view of another embodiment of a device for removing an occlusion from within the central lumen of a tubular structure according to the present invention in the low profile configuration;

FIG. 12 is a close-up lateral perspective view of the distal end of the device shown in FIG. 9 in the high profile configuration; and

FIG. 13 through FIG. 16 show partial, cutaway, lateral perspective views of various steps in a method for removing an occlusion from an occluded tubular structure according to the present invention.

### DESCRIPTION

According to one embodiment of the present invention, there is provided a device for removing an occlusion from within the central lumen of a tubular structure. The device comprises an occlusion removing structure having a low profile configuration and having a high profile configuration. The device is particularly useful for removing an occlusion from within the central lumen of a tubular structure without having to apply proximal suction, or having to move the occlusion distally. According to another embodiment of the present invention, there is provided a method for removing an occlusion from within the central lumen of a tubular structure. In one embodiment, the method comprises providing a device according to the present invention. The method further comprises inserting the device while in the low profile configuration into the tubular structure with an occlusion. Then, the device is converted to its high profile configuration, and the device is withdrawn from the tubular structure, thereby removing the occlusion from the tubular structure. The device and method of the present invention will now be disclosed in detail.

As used in this disclosure, the term “comprise” and variations of the term, such as “comprising” and “comprises,” are not intended to exclude other additives, components, integers or steps.

As used in this disclosure, the term “tubular structure” should be understood as any elongated structure having an open proximal end, a distal end and a continuously hollow central lumen extending from the open proximal end to the distal end. The distal end of the tubular structure can be either open or closed, as will be understood by those with skill in the art with reference to this disclosure. The term “tubular structure” encompasses catheters, drains, tubes such as gastrostomy and feeding tubes, and any other similar devices.

As used in this disclosure, the term “occlude” and variations of the term, such as “occluded,” “occluding,” and “occlusion” means a mass or clog of material within the central lumen of a tubular structure, which either partially or completely decreases the function of the tubular structure. As will be understood by those with skill in the art with reference to this disclosure, debris being aspirated from inside a blood vessel left after removal of an embolus or plaque from on or within the blood vessel wall by a therapy catheter, does not decrease the function of an aspiration catheter aspirating the debris, and therefore, the debris within the aspiration catheter is not an occlusion within the aspiration catheter.

All dimensions specified in this disclosure are by way of example only and are not intended to be limiting. Further, the proportions shown in these Figures are not necessarily to scale. As will be understood by those with skill in the, art with reference to this disclosure, the actual dimensions of any device or part of a device disclosed in this disclosure will be determined by its intended use.

Although the present invention is disclosed specifically with reference to its applications in the medical field; the invention can also be used in any other suitable application, including other non-medical commercial or industrial applications, as will be understood by those with skill in the art with reference to this disclosure.

The device of the present invention, and its various parts, comprises any material suitable for the purposes disclosed in this disclosure. When used in medical applications, the device will generally comprise one or more than one biocompatible material. In one embodiment, the device comprises one or more than one material that can be sterilized. In a preferred embodiment, the device consists of one or more than one biocompatible material. In another embodiment, the device comprises one or more than one material that can be discarded without creating a significant biohazard. In a preferred embodiment, the device consists of one or more than one material that can be discarded without creating a significant biohazard.

The device of the present invention, and its various parts, are manufactured according to techniques well known to those with skill in the art, as will be understood by those with skill in the art with reference to this disclosure.

According to one embodiment of the present invention, there is provided a device for removing an occlusion from within the central lumen of a tubular structure. The device comprises an occlusion removing structure having a low profile configuration and having a high profile configuration.

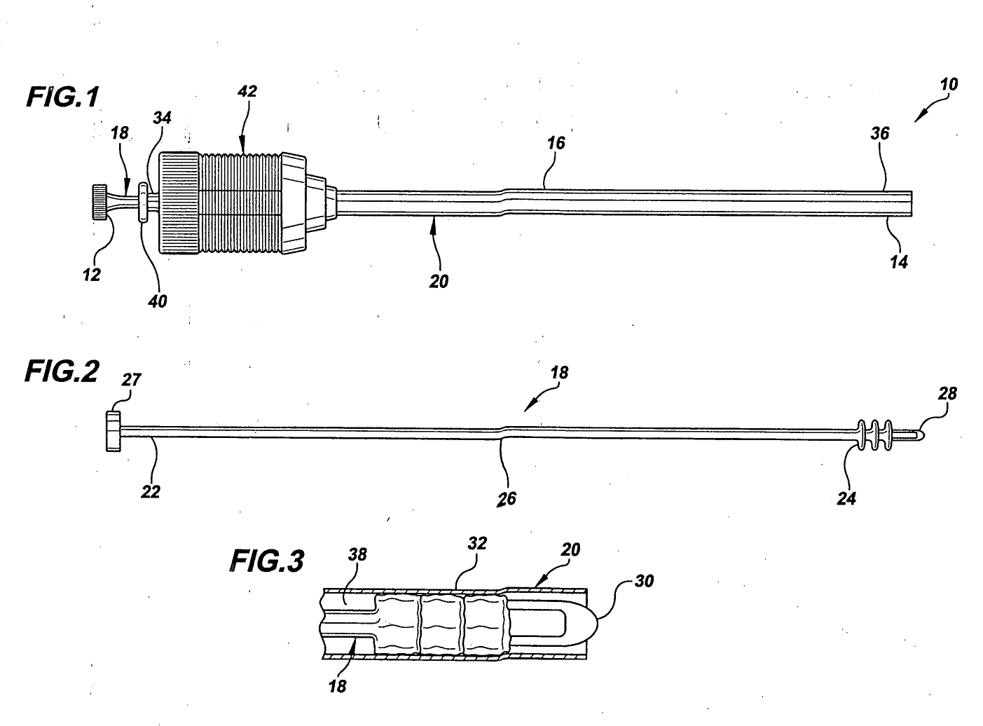

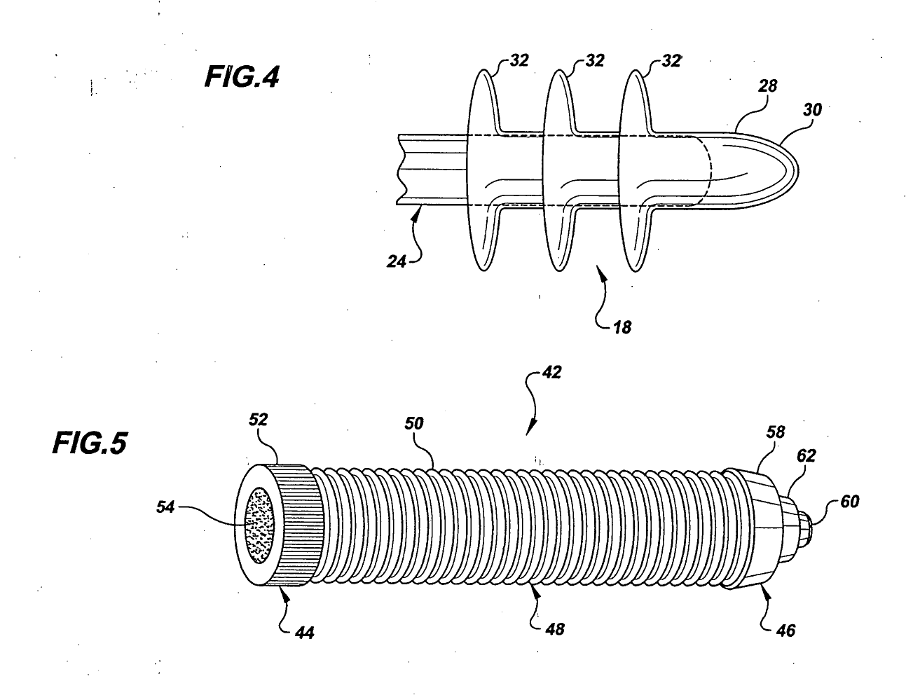

Referring now to FIG. 1 through FIG. 4, there are shown a lateral perspective view of one embodiment of a device for removing an occlusion from within the central lumen of a tubular structure according to the present invention including the optional enclosure bag (FIG. 1); a lateral perspective view of the occlusion removing structure of the device shown in FIG. 1 (FIG. 2); an enlarged cutaway lateral perspective view of the distal end of the device shown in FIG. 1 (FIG. 3); and an enlarged lateral perspective view of the distal end of the occlusion removing structure of the device shown in FIG. 1, FIG. 2 and FIG. (FIG. 4). As can be seen, the device 10 comprises a proximal end 12, a distal end 14 and an intermediate segment 16 between the proximal end 12 and the distal end 14. As shown in FIG. 1 through FIG. 4, the device 10 comprises an occlusion removing structure 18, and as shown in FIG. 1 and FIG. 3, the device 10 further comprises a sheath 20.

Referring again to FIG. 2, FIG. 3 and FIG. 4, the occlusion removing structure 18 comprises a proximal end 22, a distal end 24 and an intermediate segment 26 between the proximal end 22 and the distal end 24, and comprises an outer diameter. In a preferred embodiment, as shown in FIG. 1 and FIG. 2, the proximal end 22 of the occlusion removing structure 18 comprises a mechanism for applying axial force 27 to the distal end 24 of the occlusion removing structure 18, such as an enlarged end as shown. In one embodiment, the proximal end of the device 10 further comprises a connector, such as, for example, a screw type connector or a Luer-lock type connector.

Referring again to FIG. 2, the intermediate segment 26 of the occlusion removing structure 18 is a mechanism for transmitting axial force from the proximal end 22 of the occlusion removing structure 18 to the distal end 24 of the occlusion removing structure 18. In one embodiment, as shown, the intermediate segment 26 is a rod. In another embodiment, the intermediate segment 26 is a tube. In another embodiment, the intermediate segment 26 is a pusher wire. In a preferred embodiment, the intermediate segment 26 is significantly flexible then ordinary force is applied to the proximal end by an operator during use of the device 10 in a method according to the present invention, in order to permit the device 10 to bend around curves in the tubular structure, such as, for example, around curves of as much as 45°. The intermediate segment 26 has a longitudinal axis, and has a diameter perpendicular to the axis. In one embodiment, the axis is between about 10 cm and about 200 cm. In another embodiment, the axis is between about 20 cm and about 100 cm. In another embodiment, the axis is between about 30 cm and about 50 cm. In one embodiment, the diameter of the intermediate segment 26 is between about 0.2 mm and 4 mm. In another embodiment, the diameter of the intermediate segment 26 is between about 0.4 mm and 1 mm. In another embodiment, the diameter of the intermediate segment 26 is between about 0.5 mm and 0.6 mm.

As shown in FIG. 2, FIG. 3 and particularly FIG. 4, the distal end 24 of the occlusion removing structure 18 of the device 10 comprises an extension 28 of the intermediate segment 26. In a preferred embodiment, the extension 28 comprises a blunted tip 30 at the distal end 24 of the occlusion removing structure 18. In one embodiment, the tip 30 comprises a material selected from the group consisting of plastic, rubber and silicone. In a preferred embodiment, the tip 30 comprises a low friction material, such as, for example, polytetrafluoroethylene, that reduces friction between the tip 30 and the surface of the central lumen of the tubular structure with the occlusion.

The occlusion removing structure 18 of the device 10 further comprises one or more than one foldable disc 32 mounted transversely on the extension 28. In one embodiment, the occlusion removing structure 18 further comprises one disc 32 mounted transversely on the extension 28. In a preferred embodiment, the occlusion removing structure 18 further comprises a plurality of discs 32 mounted transversely on the extension 28. In one embodiment, the occlusion removing structure 18 further comprises two discs 32 mounted transversely on the extension 28. In another embodiment, the occlusion removing structure 18 further comprises three discs 32 mounted transversely on the extension 28. In another embodiment, the occlusion removing structure 18 further comprises four or more than four discs 32 mounted transversely on the extension 28.

Each disc 30 has a diameter and a thickness. In one embodiment, the diameter of each disc is between 0.5 mm and 20 mm. In another embodiment, the diameter of each disc is between 1 mm and 10 mm. In another embodiment, the diameter of each disc is between 2 mm and 4 mm. In one embodiment, the thickness of each disc is between 0.2 mm and 2 mm. In another embodiment, the thickness of each disc is between 0.3 mm and 1 mm. In another embodiment, the diameter of each disc is between 0.4 mm and 0.6 mm.

In one embodiment, the occlusion removing structure 18 comprises a plurality of discs 32 mounted transversely on the extension 28, and at least one of the plurality of discs 32 has a diameter different from another of the plurality of discs 32. In one embodiment, the occlusion removing structure 18 comprises at least three discs 32 mounted transversely on the extension 28, and each disc 32 is equally spaced apart from the adjacent disc 32. In one embodiment, the occlusion removing structure 18 comprises at least three discs 32 mounted transversely on the extension 28, and the distance between two adjacent discs 32 is different from the distance between two different adjacent discs 32.

In one embodiment, the occlusion removing structure 18 comprises a plurality of discs 32 mounted transversely on the extension 28, and each disc 32 is spaced apart from the adjacent disc 32 by between 0.5 mm and 10 mm. In another embodiment, the occlusion removing structure 18 comprises a plurality of discs 32 mounted transversely on the extension 28, and each disc 32 is spaced apart from the adjacent disc 32 by between 1 mm and 5 mm. In another embodiment,the occlusion removing structure 18 comprises a plurality of discs 32 mounted transversely on the extension 28, and each disc 32 is spaced a apart from the adjacent disc 32 by between 2 mm and 4 mm.

In one embodiment, each disc 32 comprises a material selected from the group consisting of plastic and rubber. In another embodiment, each disc 32 comprises a thermoplastic elastomer, such as, for example, C-Flex® (available from Cole-Palmer®, Vernon Hills, Ill. US). In a preferred embodiment, each disc comprises silicone.

Referring again to FIG. 1 and FIG. 3, the device 10 further comprises a sheath 20. The sheath 20 has a proximal end 34, a distal end 36, and a central lumen 38. The sheath 20. surrounds the intermediate segment 26 of the occlusion removing structure 18, and surrounds at east part of the distal end 24 before the device 10 is used, and during some. steps of the method according to the present invention. The sheath is axially movable with respect to the occlusion removing structure. The sheath 20 functions to fold the one or more than one disc 32 against the extension 28, thereby converting the device 10 from a high profile configuration to a low profile configuration. In one embodiment, the sheath 20 comprises a proximal collar 40 configured to prevent the proximal end 22 of the occlusion removing structure 18 from entering the central lumen 38 of the sheath 20. In a preferred embodiment, the sheath 20 is significantly flexible when ordinary force is applied to the proximal end by an operator during use of the device 10 in a method according to the present invention, in order to permit the device 10 to bend around curves in the tubular structure. In one embodiment, the sheath comprises a braided plastic sheath that resists kinking when bent. In another embodiment, the sheath 20 comprises a plastic or wire coil, however, the sheath can comprise any suitable material, as will be understood by those with skill in the art with reference to this disclosure.

In a preferred embodiment, the device 10 further comprises an enclosure bag. Referring now to FIG. 5, there is shown a lateral perspective view of an enclosure bag of the device 10 according to the present invention. As can be seen, the enclosure bag 42 comprises a proximal end 44, a distal end 46 and an intermediate segment 48. The intermediate segment 48 of the enclosure bag 42 comprises a tube 50 of material capable of being reversibly compressed and extended axially when ordinary force is applied to the proximal end 44 of the enclosure bag 42 by an operator during use of the device 10 in a method according to the present invention. The enclosure bag 42 is configured to protect the portions of the device 10 inside the enclosure bag 42 as the device 10 is introduced into a tubular structure to remove an occlusion. The tube 50 has an inner surface. When being used to remove an occlusion from a tubular structure functioning as a medical device, the inner surface of the tube 50 of the intermediate segment 48 is preferably sterile. Further, the intermediate segment 48 of the enclosure bag 42 functions to contain occluding material as the occluding material is removed from the tubular structure, as will be better understood with reference to the method of the present invention disclosed further below. In a preferred embodiment, the intermediate segment 48 of the enclosure bag 42 comprises a substantially transparent material such as, for example, a substantially transparent plastic that is sufficiently transparent to allow an operator to view the portion of the device 10 that is inside the intermediate segment 48.

In one embodiment, the proximal end 44 of the enclosure bag 42 comprises a proximal end piece 52. If present, the proximal end piece 52 comprises an axial lumen 54 having a diameter greater than the outer diameter of the occlusion removing structure 18 with the sheath 20, if present, where the diameter of the lumen 54 of the proximal end piece 52 is sufficient to allow the occlusion removing structure 18, and sheath 20 if present, to translate axially through the proximal end piece 52 and into the intermediate segment 48. In one embodiment, the proximal end piece 52 further comprises a connector, not shown.

In another embodiment, the distal end 46 of the enclosure bag 42 further comprises a distal end piece 58. If present, the distal end piece 58 also comprises an axial lumen 60 having a diameter greater than the outer diameter of the occlusion removing structure 18 and, the sheath 20 if present, where the diameter of the lumen 56 of the distal end piece 58 is sufficient to allow the occlusion removing structure 18 and, the sheath 20 if present, to translate axially from the intermediate segment 48 through the distal end piece 58 with the occluding material when the occlusion removing structure 18 is in the high profile configuration. In a preferred embodiment, the distal end piece 58 further comprises a connector 62 for mating with the proximal end of the tubular structure with an occlusion. In one embodiment, the connector 62 can be a screw type connector or a Luer-lock type connector. In a particularly preferred embodiment, the connector 62 is a “Christmas Tree” type connector. The connector 62 can, however, be any other type of connector suitable for mating with the proximal end of the tubular structure with an occlusion, as will be understood by those with skill in the art with reference to this disclosure.

In another preferred embodiment, the enclosure bag 42 further comprises a removable cover 64 (shown in FIG. 8) mating with the distal end piece 52. When present, the cover maintains the sterility of the distal end piece 52 prior to use.

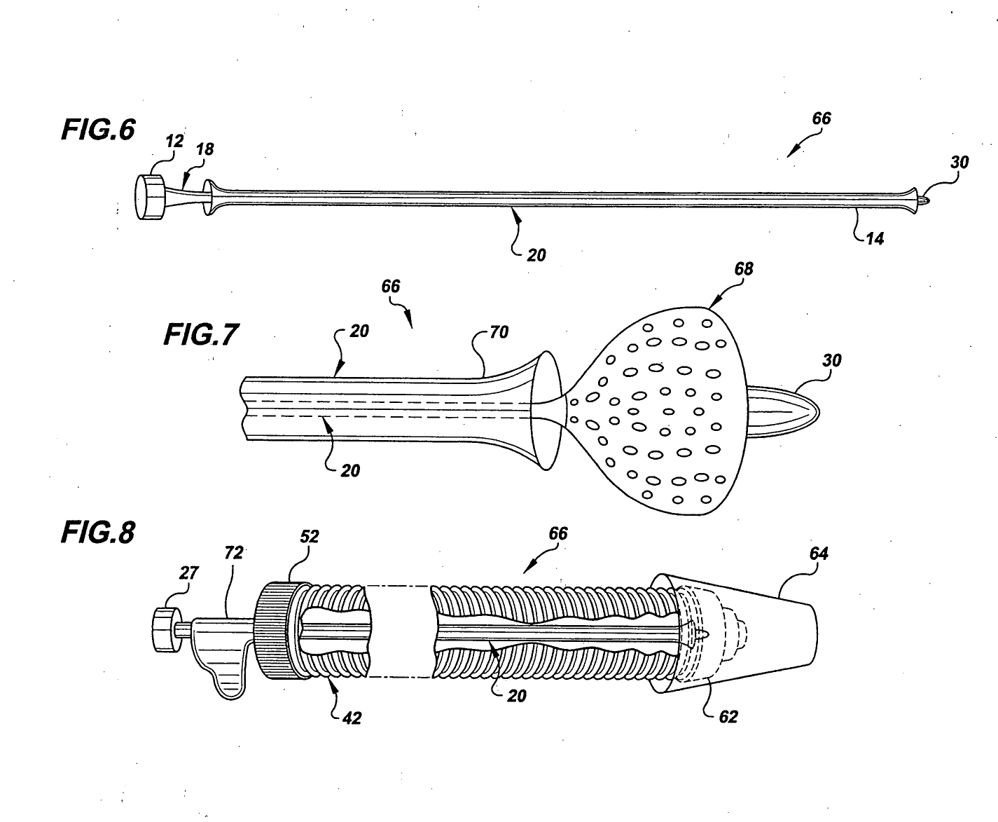

Referring now to FIG. 6 through FIG. 8, there are shown a lateral perspective view of another embodiment of a device for removing an occlusion from within the central lumen oaf tubular structure according to the present invention (FIG. 6); a partial lateral perspective view of the device shown in FIG. 6 with the distal end of the occlusion removing structure extruded from the distal end of the sheath (FIG. 7); and an enlarged cutaway partial lateral perspective view of the device shown in FIG. 6 including the optional enclosure bag (FIG. 8). As can be seen, the device 66 comprises elements corresponding to the elements disclosed and referenced with respect to the device 10. In the embodiment of the device 66, however, the distal end 24 of the occlusion removing structure 18 comprises a self-expanding portion 68 rather than one or more than one foldable disc 32. The self-expanding portion 68 is readily compressible by a compressive force, and also self-expands, that is to say, expands upon merely relieving the compressive force without either infusing a liquid or gas, or by unfolding, or by using a mechanism that untwists the structure. In this embodiment, the sheath 20 functions to compress the self-expanding portion 68 within the central lumen 38 of the sheath 20, that is, the wall of the sheath applies a compressive force to the self-expanding portion 68, thereby converting the device 66 from a high profile configuration to a low profile configuration, rather than by folding the one or more than one foldable disc 32 as with the embodiment of the device 10. In a preferred embodiment, the self-expanding portion 68 is a sponge, as shown, but the self-expanding portion 68 can be a mass of foam or of radially expanding fibers, or can be another suitable self-expanding structure, as will be understood by those with skill in the art with reference to this disclosure.

In the embodiment of the device 66, the distal end 36 of the sheath 20 further comprises a flaring distal collar 70, as shown. The distal collar 36 is configured to permit the self-expanding portion 68 of the distal end 36 of the device 66 to be retracted into the central lumen 38 of the sheath 20, as well be understood more clearly with reference to the method of the present invention.

In another preferred embodiment, the device 66 further comprises a removable tab 72 surrounding the intermediate segment 26 of the occlusion removing structure 18, immediately distal to the proximal end 14 of the device 66. The tab 72 prevents extrusion of the distal end 24 of the occlusion removing structure 18 from the distal end 36 of the sheath 20. The tab 72 comprises a reversibly tearable material, such as paper, or is perforated along a line parallel to the long axis of the intermediate segment 26 of the occlusion removing structure 18, so that the tab 72 can be removed from the intermediate segment 26, thereby permitting the distal end 24 of the occlusion removing structure 18 to extrude through the distal collar 70 of the sheath 20.

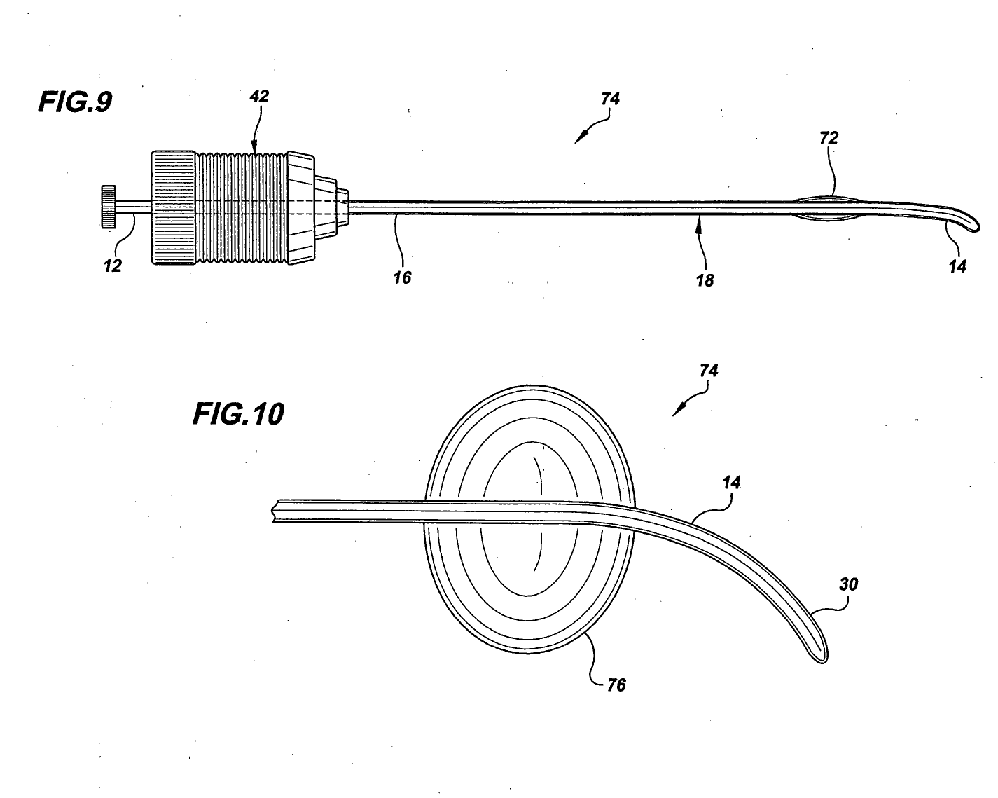

Referring now to FIG. 9 and FIG. 10, there are shown a lateral perspective view of another embodiment of a device for removing an occlusion from within the central lumen of a tubular structure according to the present invention in the low profile configuration (FIG. 9); and a close-up lateral perspective view of the distal end of the device shown in FIG. 9 in the high profile configuration (FIG. 10). As can be seen, the device 74 comprises elements corresponding to the elements disclosed and referenced with respect to the device 10 and the device 66. In the embodiment of the device 74, however, the distal end 24 of the occlusion removing structure 18 comprises an inflatable balloon 76 rather than one or more than one foldable disc 32, or a self-expanding portion 68. Further, in the embodiment of the device 74, the intermediate segment 26 of the occlusion removing structure 18 comprises a central inflation lumen that can be connected to an inflation mechanism for inflating the inflatable balloon 76. In this embodiment, the device 74 is converted from a low profile configuration to a high profile configuration by infusing a liquid or a gas through the central inflation lumen thereby inflating the inflatable balloon 76, rather than by folding the one or more than one folding disc 32 as with the embodiment of the device 10, or by relieving the compressive force on the self-expanding portion 68 as with the embodiment of the device 66. The device 74 further comprises an enclosure bag 42 as disclosed in this disclosure. The device does not require a sheath 20 to function.

In one embodiment the inflatable balloon 76 has an axial length of between 1 mm and 30 mm. In another embodiment, the inflatable balloon 76 has an axial length of between 2 mm and 20 mm. In another embodiment, the inflatable balloon 76 has an axial length of between 3 mm and 10 mm. In one embodiment, the inflatable balloon 76 has a maximum diameter of expansion of between 1 mm and 30 mm. In one, the inflatable balloon 76 comprises a material selected from the group consisting of latex, a plastic, silicone rubber, and a thermoplastic elastomer.

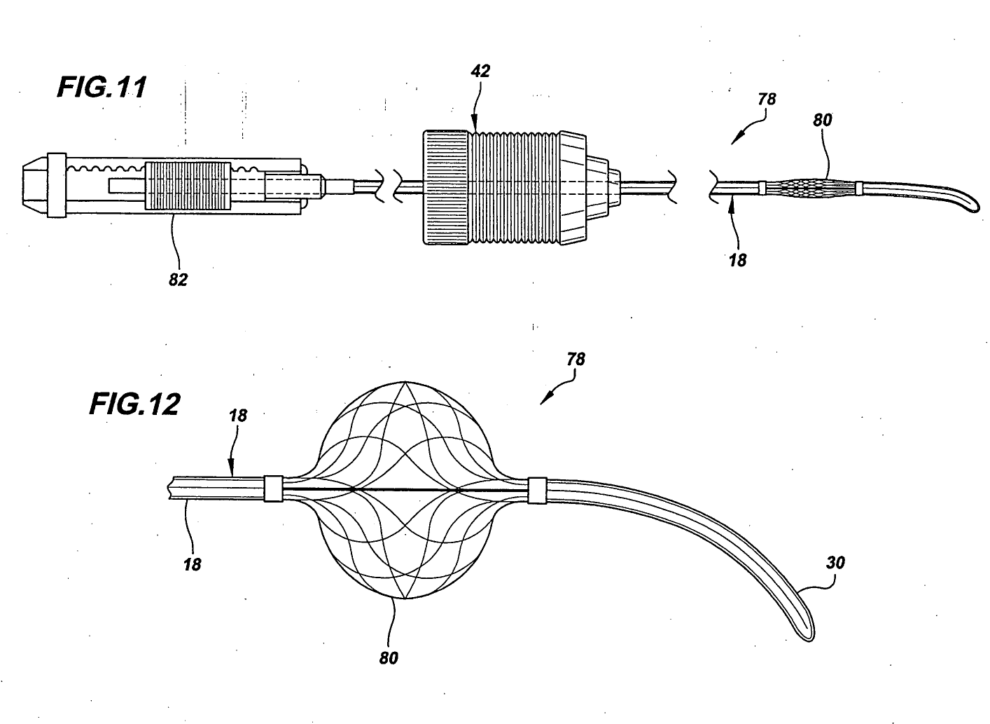

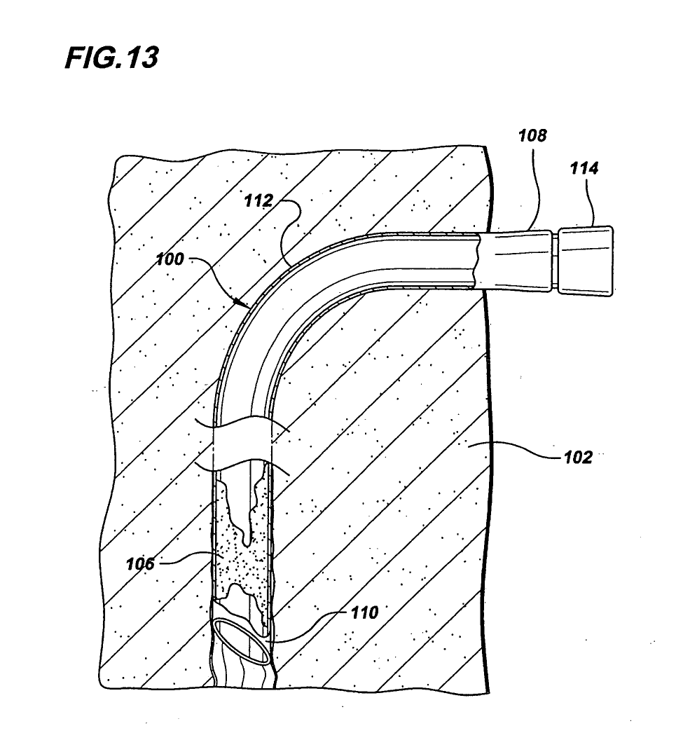

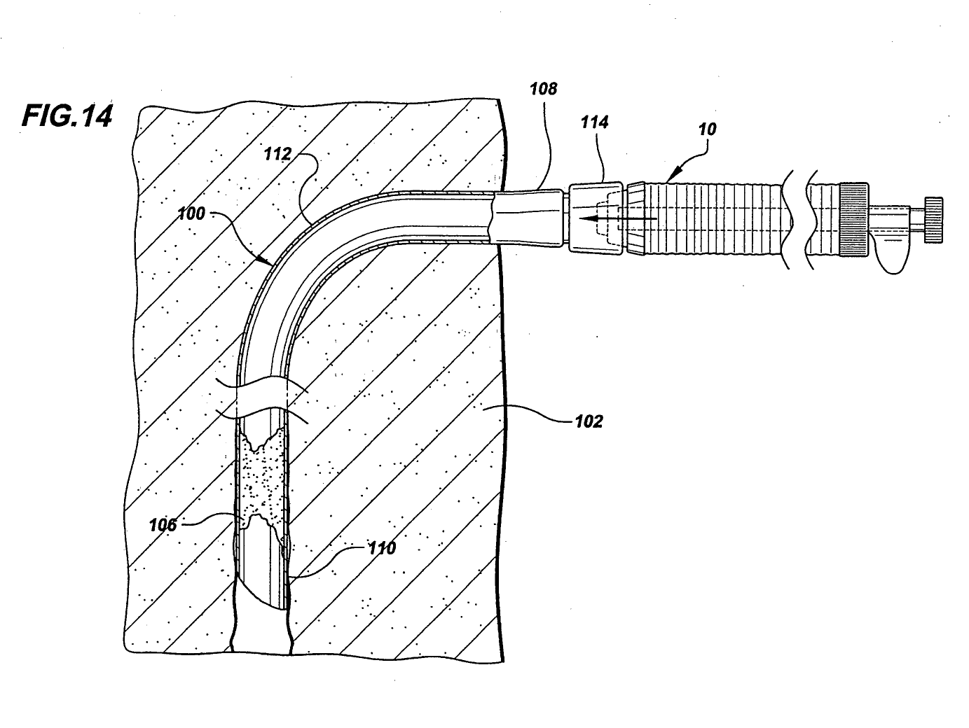

In a preferred embodiment, the intermediate segment 26 of the occlusion removing structure 18 of the device 74 comprises a braided plastic, sheath that resists kinking when bent. Referring now to FIG. 11 and FIG. 12, there are shown a lateral perspective view of another embodiment of a device for removing an occlusion from within the central lumen of a tubular structure according to the present invention in the low profile configuration (FIG. 11); and a close-up lateral perspective view of the distal end of the device shown in FIG. 11 in the high profile configuration (FIG. 12); As can be seen, the device 78 comprises elements corresponding to the elements disclosed and referenced with respect to the device 10, the device 66 and the device 74. In the embodiment of the device 72, however, the distal end 24 of the occlusion removing structure 18 comprises an expandable wire basket 80 rather than one or more than one foldable disc 32, or a self-expanding portion 68 or an inflatable balloon 76. Further, in the embodiment of the device 78, the proximal end 22 comprises a mechanism 82 for expanding the expandable wire basket 80 and the intermediate segment 26 of the occlusion removing structure 18 comprises a wire within the intermediate segment 26 connecting the mechanism 82 with the wire basket 76. In this embodiment, the device 78 is converted from a low profile configuration to a high profile configuration by activating the mechanism 82, thereby transmitting axial force to contract the wire basket 76 in a distal to proximal direction, and thereby expanding the wire basket 76 in a direction perpendicular to the axis of the device 78, rather than by folding the one or more than one foldable disc 32 as with the embodiment of the device 10, or by relieving the compressive force on a self-expanding portion 68 as with the embodiment of the device 66, or by inflating the inflatable balloon 76 as with the embodiment of the device 74. The device 78 further comprises an enclosure bag 42 as disclosed in this disclosure. The device does not require a sheath 20 to function.

According to another embodiment of the present invention, there is provided a method for removing an occlusion from within the central lumen of a tubular structure. The steps shown are not intended to be limiting nor are they intended to indicate that each step depicted is essential to the method, but instead are exemplary steps only. In one embodiment, the method comprises providing a device according to the present invention.

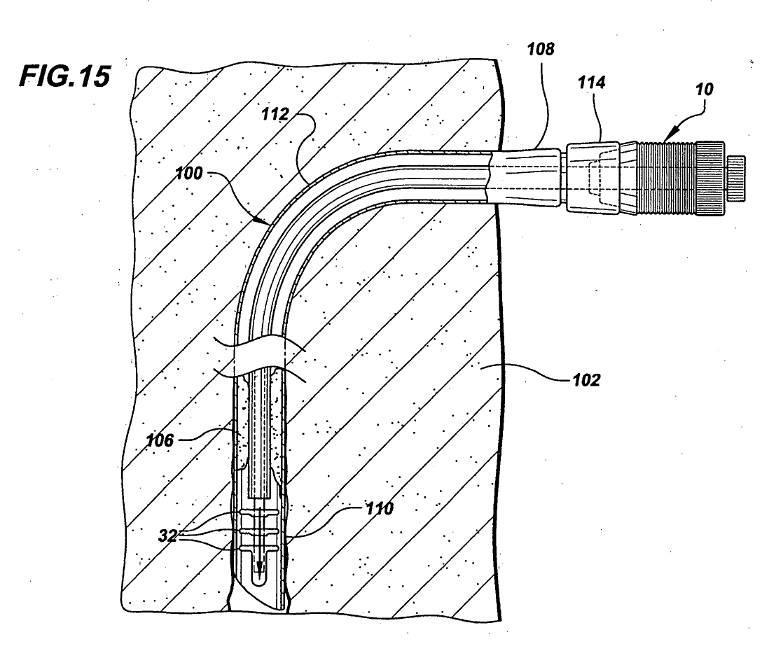

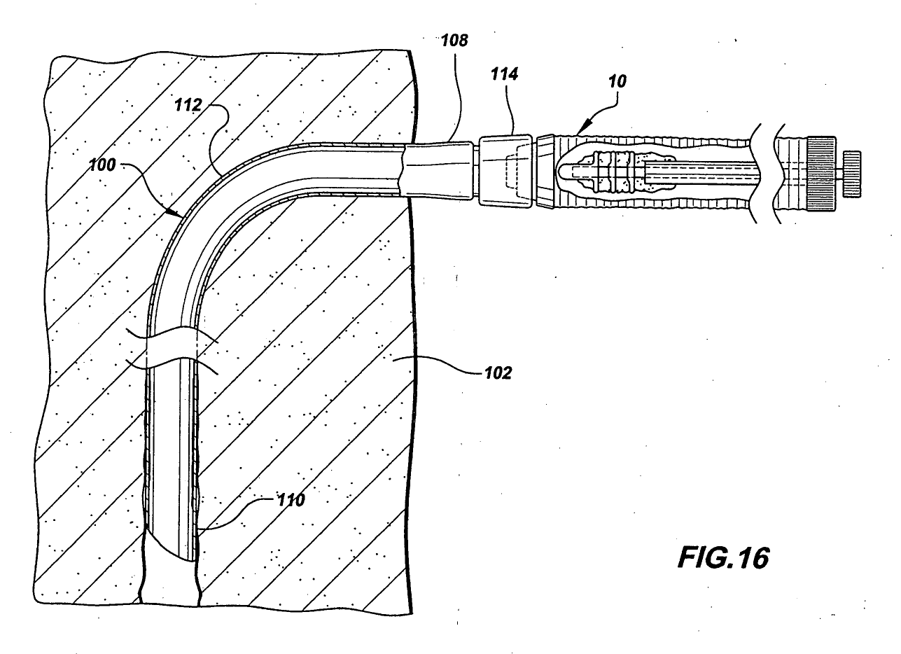

Referring now to FIG. 13 through FIG. 16, there are shown partial, cutaway, lateral perspective views of various steps in a method for removing an occlusion comprising occluding material from an occluded tubular structure according to the present invention. As can be seen in FIG. 13, a tubular structure 100 is in position, such as within a space or cavity within a human 102, and the central lumen 104 of the tubular structures 100 has either partly or completely occluded with occluding material 106. The tubular structure comprises a proximal end 108, a distal end 110, and intermediate segment 112. In a preferred embodiment, the tubular structure 100 comprises a connector 114 on the proximal end 108 as shown.

In one embodiment, the method comprises, first, providing means for removing an occlusion from within the central lumen of a tubular structure. The means for removing an occlusion comprises a proximal end and a distal end, and comprises an occlusion removing structure having a low profile configuration and having a high profile configuration. Next, the distal end of the means for removing an occlusion is inserted into the proximal end of the tubular structure in the low profile configuration. Then, the means for removing an occlusion is advanced through the central lumen of the tubular structure until the distal end of the means for removing an occlusion is at least partly distal to the occluding material. Next, the occlusion removing structure is converted into the high profile configuration. In one embodiment, converting the occlusion removing structure into the high profile configuration comprises unfolding one or more than one foldable disc, or comprises relieving compressive force on the self-expanding portion, or comprises inflating an inflatable balloon, or comprises activating a mechanism to transmit axial force to contract a wire basket in a distal to proximal direction thereby expanding the wire basket. Then, the means for removing an occlusion is withdrawn from the tubular structure while the occlusion removing structure is in the high profile configuration, thereby removing some or all of the occluding material from the tubular structure. In one embodiment, the means for removing an occlusion comprises an enclosure bag, and withdrawing the means for removing an occlusion causes the occluding material to deposit within the enclosure bag. In one embodiment, the method comprises repeating these steps. In another embodiment, the means for removing an occlusion is a first means for removing an occlusion, and method further comprises performing the same steps with a second means for removing an occlusion, where the first means for removing an occlusion and the second means for removing an occlusion comprise different mechanisms for converting the occlusion removing structure from the low profile configuration to the high profile configuration, such as, for example, where the first means for removing an occlusion is a device 10 and the second means for removing an occlusion is a device 66.

In another embodiment, the method comprises, first, providing a device according to the present invention. In a preferred embodiment, the device is a device 10, or a device 66, or a device 74 or a device 78. The device 10, 66, 70, 74 comprises a proximal end 12, a distal end 14 and an intermediate segment 16 between the proximal end 12 and the distal end 14. The device 10, 66, 70, 74 further comprises an occlusion removing structure 18 having a low profile configuration and having a high profile configuration. In a preferred embodiment, the device 10, 66 provided comprises a sheath 20. In another preferred embodiment, the device 10, 66, 70, 74 comprises an enclosure bag 42. The device 10, 66, 70, 74, the sheath 20 and the enclosure bag 42 are according to the present invention, as disclosed in this disclosure. If present, the cover 64 of the enclosure bag 42 is removed after providing the device 10, 66, 70, 74.

Referring now to FIG. 14 through FIG. 16, there are shown some steps of the method using the device 10. As can be seen, the distal end 14 of the device 10 is inserted into the proximal end 108 of the tubular structure 100 in the low profile configuration. Then, the device 10 is advanced through the central lumen 104 of the tubular structure 100 until the distal end 14 of the device 10 is at least partly distal to the occluding material 106. Next, the occlusion removing structure 18 is converted into the high profile configuration by advancing the foldable discs 32 distally relative to the distal end 36 of the sheath, thereby unfolding the discs 32. Then, the device 10 is withdrawn from the tubular structure 100 while the occlusion removing structure 18 in. the high profile configuration, thereby removing the occluding material 106 from the tubular structure 100.

As will be understood by those with skill in the art with reference to this disclosure the method depicted in FIG. 13 through FIG. 16 can also be accomplished using the device 66, where converting the occlusion removing structure 18 of the device 66 into the high profile configuration comprises relieving compressive force on the self-expanding portion 68 by advancing the self-expanding portion 68 distally relative to the distal end 36 of the sheath. The remaining steps of this embodiment of the method correspond to the steps disclosed in reference to the device 10.

As will be understood by those with skill in the art with reference to this disclosure the method depicted in FIG. 13 through FIG. 16 can also be accomplished using the device 74, where converting the occlusion removing structure 18 of the device 74 into the high profile configuration comprises inflating the inflatable balloon 76. The remaining steps of this embodiment of the method correspond to the steps disclosed in reference to the device 10.

As will be understood by those with skill in the art with reference to this disclosure the method depicted in FIG. 13 through FIG. 16 can also be accomplished using the device 78, where converting the occlusion removing structure 18 of the device 78 into the high profile configuration comprises activating the mechanism 82, thereby transmitting axial force to contract the wire basket 76 in a distal to proximal direction, and thereby expanding the wire basket 76 in a direction perpendicular to the axis of the device 78. The remaining steps of this embodiment of the method correspond to the steps disclosed in reference to the device 10.

In one embodiment of the method, the device 10, 66, 74, 78 comprises an enclosure bag 42, and inserting the distal end 14 of the device 10 into the proximal end 108 of the tubular structure 100 comprises compressing the tube 50 axially, and withdrawing the device 10, 66, 74, 78 causes the occluding material to deposit within the enclosure bag 42.

In another embodiment of the method, the proximal end 108 of the tubular structure 100 comprises a connector 114, and the method comprises connecting the connector 114 to the device 10, 66, 74, 78 through a connector on the proximal end 14 of the device 10, 66, 74, 78, or through a connector 56 on the enclosure bag 42. In one embodiment, the method further comprises unconnecting the connector 114 from the device 10, 66, 74, 78 after removing the occluding material 106.

In another embodiment of the method, the device 10, 66 comprises a sheath 20 and further comprises a removable tab 72, and the method further comprises removing the tab 72 from the intermediate segment 26, thereby permitting the distal end 24 of the occlusion removing structure 18 to extrude through the distal end 36 of the sheath.

In another embodiment, the method comprises repeating one or more than one of these steps.

In another embodiment, the device is a first device, and the method further comprises repeating the steps with a second device, where the occlusion removing structure of the first device and the occlusion removing structure of the second device are converted from the low profile configuration to the high profile configuration by two different steps selected from the group consisting of unfolding, one or more than one foldable disc, relieving compressive force on a self-expanding portion, inflating an inflatable balloon, and activating a mechanism to transmit axial force to contract a wire basket in a distal to proximal direction thereby expanding the wire basket.

Although the present invention has been discussed in considerable detail with reference to certain preferred embodiments, other embodiments are possible. Therefore, the scope of the appended claims should not be limited to the description of preferred embodiments contained in this disclosure.

1. A device for removing an occlusion from within the central lumen of a tubular structure, the device comprising:
a proximal end, a distal end and an intermediate segment between the proximal end and the distal end;
an occlusion removing structure having a low profile configuration and having a high profile configuration, the occlusion removing structure comprising a proximal end, a distal end and an intermediate segment between the proximal end and the distal end, and comprising an outer diameter.

2. The device of claim 1, where the occlusion removing structure comprises an extension of the intermediate segment;
where the occlusion removing structure further comprises one or more than one foldable disc mounted transversely on the extension, and where converting the device from the low profile configuration to the high profile configuration comprises unfolding the one or more than one disc;
where the device further comprises a sheath comprising a proximal end, a distal end, and a central lumen;
where the sheath surrounds the intermediate segment of the occlusion removing structure; and
where the sheath is axially movable with respect to the occlusion removing structure.

3. The device of claim 2, where the one or more than one foldable disc is a plurality of discs.

4. The device of claim 3, where at least one of the plurality of discs has a diameter different from another of the plurality of discs.

5. The device of claim 2, where the one or more than one foldable disc is at least three discs, and where the distance between two adjacent discs is different from the distance between two different adjacent discs.

6. The device of claim 2, where each disc comprises silicone.

7. The device of claim 1, where the device further comprises a sheath comprising a proximal end, a distal end, and a central lumen;
where the sheath is axially movable with respect to the occlusion removing structure;
where the distal end of the occlusion removing structure comprises a self-expanding portion;
where the sheath functions to compress the self-expanding portion within the central lumen of the sheath; and
where converting the device from the low profile configuration to the high profile configuration comprises relieving the compression on the self-expanding portion.

8. The device of claim 7, where the distal end of the sheath further comprises a flaring distal collar configured to permit the self-expanding portion of the distal end of the device to be retracted into the central lumen of the sheath.

9. The device of claim 2 or claim 7, further comprising a removable tab surrounding the intermediate segment of the occlusion removing structure, immediately distal to the proximal end of the device, where the tab prevents extrusion of the distal end of the occlusion removing structure from the distal end of the sheath.

10. The device of claim 2 or claim 7, where the sheath comprises a proximal collar configured to prevent the proximal end of the occlusion removing structure from entering the central lumen of the sheath.

11. The device of claim 2 or claim 7, where the occlusion removing structure further comprises a mechanism for applying axial force to the distal end of the occlusion removing structure.

12. The device of claim 2, further comprising an enclosure bag comprising a proximal end, a distal end and an intermediate segment, where the intermediate segment of the enclosure bag comprises a tube of material capable of being reversibly compressed and extended axially.

13. The device of claim 1, where the distal end of the occlusion removing structure comprises an inflatable balloon, and where converting the device from the low profile configuration to the high profile configuration comprises inflating the balloon; and
where the device further comprises an enclosure bag comprising a proximal end, a distal end and an intermediate segment, where the intermediate segment of the enclosure bag comprises a tube of material capable of being reversibly compressed and extended axially.

14. The device of claim 1, where the device has a long axis;
where the distal end of the occlusion removing structure comprises an expandable wire basket, and where converting the device from the low profile configuration to the high profile configuration comprises activating a mechanism, thereby transmitting axial force to contract the wire basket in a distal to proximal direction, and thereby expanding the wire basket in a direction perpendicular to the axis of the device; and
where the device further comprises an enclosure bag comprising a proximal end, a distal end and an intermediate segment, where the intermediate segment of the enclosure bag comprises a tube of material capable of being reversibly compressed and extended axially.

15. The device of claim 12 or claim 13 or claim 14 or claim 41, where the intermediate segment of the enclosure bag comprises a substantially transparent material.

16. The device of claim 12 or claim 13 or claim 14 or claim 41, where the proximal end of the enclosure bag comprises a proximal end piece comprising an axial lumen having a diameter greater than the outer diameter of the occlusion removing structure.

17. The device of claim 12 or claim 13 or claim 14 or claim 41, where the distal end of the enclosure bag comprises a distal end piece comprising an axial lumen having a diameter greater than the outer diameter of the occlusion removing structure.

18. The device of claim 12 or claim 13 or claim 14 or claim 41, where the distal end of the enclosure bag comprises a connector for mating with the proximal end of the tubular structure with an occlusion.

19. The device of claim 17, where the connector is a “Christmas Tree” type connector or is a Luer-lock type connector.

20. The device of claim 12 or claim 13 or claim 14 or claim 41, where the enclosure bag further comprises a removable cover.

21. The device of claim 2 or claim 7 or claim 13 or claim 14, where the proximal end of the device further comprises a connector.

22. The device of claim 2 or claim 7 or claim 13 or claim 14, where the occlusion removing structure comprises an extension of the intermediate segment, and where the extension comprises a blunted tip at the distal end of the occlusion removing structure.

23. A method for removing an occlusion from a tubular structure, where the tubular structure comprises a proximal end, a distal end, and intermediate segment and a central lumen within the intermediate segment, and where the occlusion is within the central lumen, the method comprising:
a) providing means for removing an occlusion from within the central lumen of a tubular structure, where the means for removing an occlusion comprises a proximal end and a distal end, and comprises an occlusion removing structure having a low profile configuration and having a high profile configuration;
b) inserting the distal end of the means for removing an occlusion into the proximal end of the tubular structure in the low profile configuration;
c) advancing the means for removing an occlusion through the central lumen of the tubular structure until the distal end of the means for removing an occlusion is at least partly distal to the occluding material;
d) converting the occlusion removing structure into the high profile configuration; and
e) withdrawing the means for removing an occlusion from the tubular structure while the occlusion removing structure is in the high profile configuration, thereby removing some or all of the occluding material from the tubular structure.

24. The method of claim 23, where the means for removing an occlusion comprises one or more than one foldable disc, and where converting the occlusion removing structure into the high profile configuration comprises unfolding the one or more than one foldable disc.

25. The method of claim 23, where the means for removing an occlusion comprises a self-expanding portion, and where converting the occlusion removing structure into the high profile configuration comprises relieving compressive force on the self-expanding portion.

26. The method of claim 24 or claim 25, where the means for removing an occlusion comprises an enclosure bag; and
withdrawing the means for removing an occlusion causes the occluding material to deposit within the enclosure bag.

27. The method of claim 23, where the means for removing an occlusion comprises an inflatable balloon;
where converting the occlusion removing structure into the high profile configuration comprises inflating the inflatable balloon;
where the means for removing an occlusion comprises an enclosure bag; and
withdrawing the means for removing an occlusion causes the occluding material to deposit within the enclosure bag.

28. The method of claim 23, where the means for removing an occlusion comprises a wire basket;
where converting the occlusion removing structure into the high profile configuration comprises activating a mechanism to transmit axial force to contract a wire basket in a distal to proximal direction thereby expanding the wire basket;
where the means for removing an occlusion comprises an enclosure bag; and
withdrawing the means for removing an occlusion causes the occluding material to deposit within the enclosure bag.

29. The method of claim 23, comprising repeating steps b) through e).

30. The method of claim 23, where the means for removing an occlusion is a first means for removing an occlusion, and where the method further comprises performing the steps b) through e) with a second means for removing an occlusion, where the first means for removing an occlusion and the second means for removing an occlusion comprise different mechanisms for converting the occlusion removing structure from the low profile configuration to the high profile configuration.

31. The method of claim 23, where the tubular structure comprises a connector on the proximal end, and where the method further comprises connecting the means for removing an occlusion to the connector.

32. A method for removing an occlusion from a tubular structure, where the tubular structure comprises a proximal end, a distal end, an intermediate segment and a central lumen within the intermediate segment, and where the occlusion is within the central lumen, the method comprising:
a) providing a device according to claim 2 or claim 7;
b) inserting the distal end of the device into the proximal end of the tubular structure in the low profile configuration;
c) advancing the device through the central lumen of the tubular structure until the distal end of the device is at least partly distal to the occluding material;
d) converting the occlusion removing structure into the high profile configuration; and
e) withdrawing the device from the tubular structure while the occlusion removing structure is in the high profile configuration, thereby removing some or all of the occluding material from the tubular structure.

33. The method of claim 32, where the device comprises an enclosure bag, and withdrawing the device causes the occluding material to deposit within the enclosure bag.

34. The method of claim 32, where the device further comprises a removable tab; and
where the method further comprises removing the tab from the intermediate segment, thereby permitting the distal end of the occlusion removing structure to extrude through the distal end of the sheath.

35. A method for removing an occlusion from a tubular structure, where the tubular structure comprises a proximal end, a distal end, an intermediate segment and a central lumen within the intermediate segment, and where the occlusion is within the central lumen, the method comprising:
a) providing a device according to claim 13 or claim 14;
b) inserting the distal end of the device into the proximal end of the tubular structure in the low profile configuration;
c) advancing the device through the central lumen of the tubular structure until the distal end of the device is at least partly distal to the occluding material;
d) converting the occlusion removing structure into the high profile configuration; and
e) withdrawing the device from the tubular structure while the occlusion removing structure is in the high profile configuration, thereby removing some or all of the occluding material from the tubular structure.

36. The method of claim 35, where withdrawing the device causes the occluding material to deposit within the enclosure bag.

37. The method of claim 32, comprising repeating steps b) through e).

38. The method of claim 32, where the device is a first device, and where the method further comprises performing the steps b) through e) with a second device, where the first device and the second device comprise different mechanisms for converting the occlusion removing structure from the low profile configuration to the high profile configuration.

39. The method of claim 32, where the tubular structure comprises a connector on the proximal end, and where the method further comprises connecting the device to the connector.

40. The method of claim 39, where the method further comprises unconnecting the connector from the device after removing the occluding material.

41. The device of claim 7, further comprising an enclosure bag comprising a proximal end, a distal end and an intermediate segment, where the intermediate segment of the enclosure bag comprises a tube of material capable of being reversibly compressed and extended axially.

42. The method of claim 35, comprising repeating steps b) through e).

43. The method of claim 35, where the device is a first device, and where the method further comprises performing the steps b) through e) with a second device, where the first device and the second device comprise different mechanisms for converting the occlusion removing structure from the low profile configuration to the high profile configuration.

44. The method of claim 35, where the tubular structure comprises a connector on the proximal end, and where the method further comprises connecting the device to the connector.
